SEQUENCE LISTING (10 sequences)

SEQ ID NO: 1 - DNA, 3819 bp, Arabidopsis thaliana
tgctggaacgaatcttcttagccctccttcttctactgacgaccttcttactcttctcgatgtacactttcttcatcgttgcttttgctcaaatagattctcttaggtttggattcacggaatgcgattagggtttctctttcttgccgataaggaaaacttgattgttgattttagtcatatgtttgatcaaatctgggtgctttcttctgaattcgttgcgttttcttgtctatccaacttgttaatttggttaaaggcgaatccttttcttctaattcaattgtctctggcgtaggaaactgagtctctgcttaaaaatgtggagcaagatcaaccattatcaatgcaaagtgccctaattccatccaggaatgctttggtatcagttgaccttttaagccatcctgattctgatgttagggtttcagttgtctcttgcttaaccgagattgtgaggattactgccccagaaaccccttacagtgatgatctaatgaaggtaatatcaaatttcactaacactgtcttcaatgtcactgtctctctctctttactttatctgttggttcctacttctttggtatcacaggagatcttcaggttgacaatagaagctttcgagaaattagctgatgcctcctctcggagttataagaaagccgagtttgttcttgataatgttgcaaaggtcaaatcgtgtttggtgatgttggacttggaatgctatgacctcatcctacaaatgtttcggaacttcttcaaattcataaggtagattagatatacaaaaagaccttatttacaatacttgagacaaactctttagattatagactgaatgggggttcttttgggtgttcatctaatagatctgatcatcctcaactggtcttttcgtcaatggaattaataatgattgcaataatagatgaaaccgaacaagtgtccacggatttgcttgatagtctcttagcaactgtcaaaaaggaaaatcaggtaaggtttcttcttatttcaagttaattatctcgcagtcaatgaacttgggattttgatttttattctcttcctgtcttagaatgtttcaccaatgtcttggagtcttgcggagaaggttcttagtagatgtgctcgtaaacttaaaccatacatcatcgaagctttgaagtctagagggaccagcttggatatgtactctccagtagtttcgtccatatgccagagtgtttttaacactcctaaagtccacagtccagttaacaccaaagaacatgaggtattatatttggcgagcttgttcatttgtagagtttcagcatcttttaatagtgtcgtttaaccaaataccttgatctaggagaaattggatttggggcattctcgcaaggagaatctttctaaaagtagttccaagagacctgcaagacatgaaactagaggaatcaatgagaaggaaaaagttagaaacggaaacaaatctagtttgttgaaacagagtctgaagcaagtgaggtctgaaagtacagatgcagaaataacagggaagagaggacggaaacccaattctttaatgaatcctgaggattatgacatttcttggctttcaggaaaaagagatcctttaaagacgtcttcaaacaaaaagatccagaaaaaaggatctgggggagtatcatcactaggaaaggtgcctgccaagaaaacacctttacctaaagaaaattccccagccacgagtagtaggtctctgacgggttcacttaaacgaagccgggttaagatggatgagagtgactatgattctgattctctttcttcaccgagattgaagaaattggcatcatgcttccgggatgaagagccaaaccaagaagatgacagaaagattggaaactccagcaaacagactaggtccaaaaatggtttagagaagagtcagaaaacagccaagaagaagccagttgtagaagctaagattgtaaactccagtgggaagagactatcagctcgctcggttgctaagagaaggaatttagaacgtgcacccctagatactcttgttccacaatcatcaaagagaaaggttgaaaacaagacagacgatcatagatttctcttgtcgaaataatactgttaaaccttttgttgaatttcacgtttggatcaactgtgcagaagatggtttctcaagttgcagctagacaattggccaacgaatcagaagaagaaactccaaagagccatccgacaaggagacggacagtgagaaaagaagtggtataataagctttgttaccttctctccccatttttagccattgattgtcacctatctgttaccatgtgacatatggatttccatcttttaaggagtctgatggctttggcgaggatttggtcggtaagagagtcaatatctggtggccgctcgacaagacgtaagtgtattggaaacttgaaggttcttatttccaagtgtactgtaatccttgtttttccgttgatggtcttacactgtgcagattttatgaaggcgtcatagattcctattgtactcgtaagaagatgcatcgggtgagagaatatctctgatctgctattcagttctgttcctcctatcagaatcgtgcctgtttcttaattgattgatgtggaatgtttgttcccccactggttgcaggtaatatattctgatggagattccgaagagcttaatctcactgaagagcgctgggagttactcgaggatgacacttcggccgatgaggtacaagtttcttctatttgttttggaataaagtgtaatcgccgtgcttaatgattttcccacaatcgatcagcaggataaggagattgatctgccagagtccattcctttatctgacatgtgagtaaatcggttcattactgtgatctgtgtaaagttgcaatcttgatcttctatggtattaaaggtaatagtctattccggttcttatgatgttgcagaatgcagaggcagaaagttaagaaaagcaaaaacgtggcagtgtctgtggaaccgactagttcctcaggtgtaaggtgtgtgagaatttactaaaattcaagttattgtttatatgaaattttgatgatgacttgttctgagaggattggcgtgtatattgatggtgatagatcctcaagtagaacacttatgaagaaggattgtggcaaaaggttgaataaacaagttgaaaaaacaagagaaggaaagaatctaagatcgttaaaagagttgaatgctgaaactgacaggacagcagaagagcaggaagtgagtctagaagctgaatctgatgacagaagcgaagagcaggaatacgaagatgattgtagcgataagaaagaacaatctcaggacaaaggtgtagaggctgaaactaaggaagaagagaaacaatatccaaattcagagggtgagagtgaaggagaggactcagagtcagaggaagagccgaaatggagagaaacagatgatatggaggatgatgaagaagaagaagaagaagagattgatcatatggaggatgaagcagaagaagagaaagaagaggttgatgataaagaggcaagcgcaaacatgtctgagattgagaaagaagaagaagaagaagaagaagatgaagagaagagaaagtcatgaaggagttacatagagttagagcattgtaagctaaaaccatttcagaaagattctttctgcttagacgctctggtttatctttcttagtagatttgttgatattgaaccaagttttagatgaggtcacctggtttgtgtttgtgtcttga

SEQ ID NO: 2 - DNA, 21 bp, Artificial Sequence
  SALK_009831 Left Primer
ttccattgacgaaaagaccag

SEQ ID NO: 3 - DNA, 21 bp, Artificial Sequence
  SALK_009831 Right Primer
gaatcacccgaaagctctctc

SEQ ID NO: 4 - DNA, 21 bp, Artificial Sequence
  SALK_133849 Left Primer
agaaccttctccgcaagactc

SEQ ID NO: 5 - DNA, 21 bp, Artificial Sequence
  SALK_133849 Right Primer
tgttggatttgaccagctttc

SEQ ID NO: 6 - DNA, 19 bp, Artificial Sequence
  SALK T-DNA insert LBb1.3
attttgccgatttcggaac

SEQ ID NO: 7 - DNA, 20 bp, Arabidopsis thaliana
tttcttctaattcaattgtc

SEQ ID NO: 8 - DNA, 20 bp, Arabidopsis thaliana
attctgatgtcttaaccgag

SEQ ID NO: 9 - DNA, 10 bp, Artificial Sequence
  Partial, interrupted T-DNA insertion for lrt1-1 SALK_009831
caaatcgctg

SEQ ID NO: 10 - DNA, 10 bp, Artificial Sequence
  Partial, interrupted T-DNA insertion for lrt1-2 SALK_133849
gtagaataat

(19) United States
(12) Patent Application Publication
(10) Pub. No.: US 2022/0290171 A1
(43) Pub. Date: Sep. 15, 2022

(54) METHODS FOR INCREASING TISSUE STORAGE LIPIDS BY DISRUPTING PLANT LIPID REGULATORY SUPPRESSOR GENE

(71) Applicant: University of North Texas, Denton, TX (US)

(72) Inventors: Kent Chapman, Denton, TX (US); Ann Price, Denton, TX (US); Kevin Mutore, Denton, TX (US)

(73) Assignee: University of North Texas, Denton, TX (US)

(21) Appl. No.: 17/678,269

(22) Filed: Feb. 23, 2022

Related U.S. Application Data
(60) Provisional application No. 63/160,190, filed on Mar. 12, 2021.

(51) Int. Cl. C12N 15/82; C07K 14/415

(52) U.S. Cl. CPC C12N 15/8247; C07K 14/415; C12N 15/8251

(57) ABSTRACT

Disruption of a Lipid Droplet Regulatory—Tudor Domain Containing (LRT1) gene in plants leads to accumulation of storage lipids in cells of the plants. An increased amount of lipids storage in these plants offers new possibilities for developing crops with more energy dense biomass and increased seed oil content.

This application claims priority to U.S. Provisional Patent Application Ser. No. 63/160,190 entitled “Methods for Increasing Tissue Storage Lipids by Disrupting Plant Lipid Regulatory Suppressor Gene,” filed Mar. 12, 2021, the entire contents of which are hereby incorporated by reference.

### BACKGROUND

This disclosure pertains to methods for producing plants having accumulated lipids.

Plants convert light energy into usable chemical energy by the process of photosynthesis. Under sufficient light energy, plant leaves will store excess chemical energy in the form of starch inside leaf chloroplasts, or depending upon the physiological needs of the plant, will transport carbohydrates to other tissues for growth or storage. During reproductive stages of plant development, this carbon originally captured by photosynthesis is often converted into more reduced and energy-dense lipid soluble molecules for efficient storage in seeds and fruits. These storage lipids are the familiar vegetable oils found in oilseeds (like soybean and canola) and oleaginous fruits (like oil palm and avocado).

Leaf tissues in plants rarely accumulate and store large quantities of lipids primarily due to the differences in metabolic programming from that found in seeds (Chapman and Ohlrogge, 2012; Chapman et al., 2013). However, there is considerable interest in the energy-densification of plant biomass more broadly for bioenergy and nutritional applications, and one way to do this would be to divert the metabolic pathways in leaves toward oil biosynthesis like those found oil-storing tissues. Indeed, there have been significant advances in this area where leaves of tobacco plants were engineered to accumulate oil at more than 30% by weight (from normally less than 0.5%). Generally in these metabolic engineering strategies, genes are introduced to “push, pull, package and protect” synthesized oils in the cytoplasm of leaf cells (Vanhercke et al., 2019). In nearly all of these metabolic engineering reports to date, the introduction of genes or upregulation of their expression is required to direct oil accumulation in leaves. What is lacking are strategies where the loss-of-function of genes results in oil accumulation, a strategy that would be more easily amenable to non-GMO approaches.

### SUMMARY

The present disclosure relates generally to methods for producing plants having increased accumulation of lipids by disrupting the plants' lipid regulatory suppressor gene.

In particular, the present disclosure relates to an identified plant gene that when disrupted in Arabidopsis, leads to proliferation of lipid droplets and storage lipid accumulation in leaves and seeds, suggesting it normally functions to suppress lipid accumulation. Two independent mutant alleles, lrt1-1 and lrt1-2, with T-DNA disruptions at different locations in the gene both show a proliferation of cytoplasmic lipid droplets in leaves as well as increased triacylglycerol (storage oil) content. The protein encoded by this gene normally localizes to the nucleus and has a predicted domain organization similar to proteins known to interact with and remodel chromatin. This protein likely normally suppresses the accumulation of storage lipids in plant tissues, and its loss-of-function results in the production of storage lipids in tissues that is most evident where they normally do not occur. Mutant plants were normal in all respects of growth, development and photosynthesis, although they appeared to flower at a significantly earlier time point. Seeds of these mutants were significantly larger and also had significantly higher amounts of storage lipids. Overall an increased amount of lipids storage in these plants offers new possibilities for developing crops with more energy dense biomass and increased seed oil content—satisfying both bioenergy and nutritional needs.

### BRIEF DESCRIPTION OF THE DRAWINGS

FIG. 1A shows an intron, exon map of the LRT1 gene showing the locations of the T-DNA inserts.

FIG. 1B shows sequences of the insertion sites for lrt1-1 SALK_009831—TTTCTTCTAATTCAATTGTC (SEQ ID NO.7)—and lrt1-2 SALK_133849—ATTCTGATGTCTTAACCGAG (SEQ ID NO.8)—where white boxes indicate the T-DNA insertions shown with partial, interrupted sequences CAAAT . . . CGCTG (SEQ ID NO:9) and GTAGA . . . ATAAT (SEQ ID NO:10).

FIG. 1C shows a PCR analysis showing the presence of the T-DNA inserts in the genomic DNA of the mutant lines.

FIG. 1D shows RT-PCR showing expression of full length LRT1 in WT tissues, but none in SALK mutant lines.

FIG. 2 shows confocal micrographs of representative images of leaf areas of 28 day old plants.

FIG. 3A shows quantification of increase in lipid droplet area in mutant leaves monitored by fluorescence microscopy.

FIG. 3B shows a mass spec analysis where each bar represents quantities of lipids obtained from 3 biological replicates.

FIG. 4 shows profiles of storage lipid triacylglycerol (TAG) individual molecular species in the mutant plants.

FIG. 5A shows single layer confocal laser scanning microscopy (CLSM) images of stained seed sections of embryos showing lipid droplets in lrt mutants versus wild-type.

FIG. 5B shows Airyscan CLSM images of stained seed sections of embryos showing lipid droplets in lrt mutants versus wild-type.

FIG. 5C shows (Left) graph showing the weight of 100 seeds, (Middle) seed oil content measured by time-domain, and (Right) total seed weight, for lrt mutants and wild-type.

FIG. 6A shows confocal images of portions of leaves stained to show lipid droplets with chloroplasts marked by autofluorescence.

FIG. 6B shows a graph quantifying lipid droplets in multiple images from cotyledons during 28 days of development.

FIG. 6C and 6D show graphs quantifying lipid droplets in representative true leaves during 28 days of development.

FIG. 7A shows total photosynthetic leaf areas quantified over 28 days of development for wild type and the two mutant plants.

FIG. 7B shows photographs of wild type and mutant plants over 28 days.

FIG. 8A shows days to bolting (<1 cm) for wild type and mutant plants.

FIG. 8B shows days to first flower opening for wild type and mutant plants.

FIG. 9 shows photosynthetic rate calculated for wild type and both mutant plants.

### DETAILED DESCRIPTION OF PREFERRED EMBODIMENTS

The present disclosure relates to methods for producing plants that accumulate and store lipids in their tissues in increased amounts by disrupting expression of a lipid regulatory suppressor gene.

The Arabidopsis gene locus, At1g80810 (designated LRT1), consists of 13 exons and 12 introns, and is predicted to encode a protein of 773 amino acids. The sequence of At1g80810 is shown below:

(SEQ ID NO: 1)
TGCTGGAACG AATCTTCTTA GCCCTCCTTC TTCTACTGAC
GACCTTCTTA CTCTTCTCGA TGTACACTTT CTTCATCGTT
GCTTTTGCTC AAATAGATTC TCTTAGGTTT GGATTCACGG
AATGCGATTA GGGTTTCTCT TTCTTGCCGA TAAGGAAAAC
TTGATTGTTG ATTTTAGTCA TATGTTTGAT CAAATCTGGG
TGCTTTCTTC TGAATTCGTT GCGTTTTCTT GTCTATCCAA
CTTGTTAATT TGGTTAAAGG CGAATCCTTT TCTTCTAATT
CAATTGTCTC TGGCGTAGGA AACTGAGTCT CTGCTTAAAA
ATGTGGAGCA AGATCAACCA TTATCAATGC AAAGTGCCCT
AATTCCATCC AGGAATGCTT TGGTATCAGT TGACCTTTTA
AGCCATCCTG ATTCTGATGT TAGGGTTTCA GTTGTCTCTT
GCTTAACCGA GATTGTGAGG ATTACTGCCC CAGAAACCCC
TTACAGTGAT GATCTAATGA AGGTAATATC AAATTTCACT
AACACTGTCT TCAATGTCAC TGTCTCTCTC TCTTTACTTT
ATCTGTTGGT TCCTACTTCT TTGGTATCAC AGGAGATCTT
CAGGTTGACA ATAGAAGCTT TCGAGAAATT AGCTGATGCC
TCCTCTCGGA GTTATAAGAA AGCCGAGTTT GTTCTTGATA
ATGTTGCAAA GGTCAAATCG TGTTTGGTGA TGTTGGACTT
GGAATGCTAT GACCTCATCC TACAAATGTT TCGGAACTTC
TTCAAATTCA TAAGGTAGAT TAGATATACA AAAAGACCTT
ATTTACAATA CTTGAGACAA ACTCTTTAGA TTATAGACTG
AATGGGGGTT CTTTTGGGTG TTCATCTAAT AGATCTGATC
ATCCTCAACT GGTCTTTTCG TCAATGGAAT TAATAATGAT
TGCAATAATA GATGAAACCG AACAAGTGTC CACGGATTTG
CTTGATAGTC TCTTAGCAAC TGTCAAAAAG GAAAATCAGG
TAAGGTTTCT TCTTATTTCA AGTTAATTAT CTCGCAGTCA
ATGAACTTGG GATTTTGATT TTTATTCTCT TCCTGTCTTA
GAATGTTTCA CCAATGTCTT GGAGTCTTGC GGAGAAGGTT
CTTAGTAGAT GTGCTCGTAA ACTTAAACCA TACATCATCG
AAGCTTTGAA GTCTAGAGGG ACCAGCTTGG ATATGTACTC
TCCAGTAGTT TCGTCCATAT GCCAGAGTGT TTTTAACACT
CCTAAAGTCC ACAGTCCAGT TAACACCAAA GAACATGAGG
TATTATATTT GGCGAGCTTG TTCATTTGTA GAGTTTCAGC
ATCTTTTAAT AGTGTCGTTT AACCAAATAC CTTGATCTAG
GAGAAATTGG ATTTGGGGCA TTCTCGCAAG GAGAATCTTT
CTAAAAGTAG TTCCAAGAGA CCTGCAAGAC ATGAAACTAG
AGGAATCAAT GAGAAGGAAA AAGTTAGAAA CGGAAACAAA
TCTAGTTTGT TGAAACAGAG TCTGAAGCAA GTGAGGTCTG
AAAGTACAGA TGCAGAAATA ACAGGGAAGA GAGGACGGAA
ACCCAATTCT TTAATGAATC CTGAGGATTA TGACATTTCT
TGGCTTTCAG GAAAAAGAGA TCCTTTAAAG ACGTCTTCAA
ACAAAAAGAT CCAGAAAAAA GGATCTGGGG GAGTATCATC
ACTAGGAAAG GTGCCTGCCA AGAAAACACC TTTACCTAAA
GAAAATTCCC CAGCCACGAG TAGTAGGTCT CTGACGGGTT
CACTTAAACG AAGCCGGGTT AAGATGGATG AGAGTGACTA
TGATTCTGAT TCTCTTTCTT CACCGAGATT GAAGAAATTG
GCATCATGCT TCCGGGATGA AGAGCCAAAC CAAGAAGATG
ACAGAAAGAT TGGAAACTCC AGCAAACAGA CTAGGTCCAA
AAATGGTTTA GAGAAGAGTC AGAAAACAGC CAAGAAGAAG
CCAGTTGTAG AAGCTAAGAT TGTAAACTCC AGTGGGAAGA
GACTATCAGC TCGCTCGGTT GCTAAGAGAA GGAATTTAGA
ACGTGCACCC CTAGATACTC TTGTTCCACA ATCATCAAAG
AGAAAGGTTG AAAACAAGAC AGACGATCAT AGATTTCTCT
TGTCGAAATA ATACTGTTAA ACCTTTTGTT GAATTTCACG
TTTGGATCAA CTGTGCAGAA GATGGTTTCT CAAGTTGCAG
CTAGACAATT GGCCAACGAA TCAGAAGAAG AAACTCCAAA
GAGCCATCCG ACAAGGAGAC GGACAGTGAG AAAAGAAGTG
GTATAATAAG CTTTGTTACC TTCTCTCCCC ATTTTTAGCC
ATTGATTGTC ACCTATCTGT TACCATGTGA CATATGGATT
TCCATCTTTT AAGGAGTCTG ATGGCTTTGG CGAGGATTTG
GTCGGTAAGA GAGTCAATAT CTGGTGGCCG CTCGACAAGA
CGTAAGTGTA TTGGAAACTT GAAGGTTCTT ATTTCCAAGT
GTACTGTAAT CCTTGTTTTT CCGTTGATGG TCTTACACTG
TGCAGATTTT ATGAAGGCGT CATAGATTCC TATTGTACTC
GTAAGAAGAT GCATCGGGTG AGAGAATATC TCTGATCTGC
TATTCAGTTC TGTTCCTCCT ATCAGAATCG TGCCTGTTTC
TTAATTGATT GATGTGGAAT GTTTGTTCCC CCACTGGTTG
CAGGTAATAT ATTCTGATGG AGATTCCGAA GAGCTTAATC
TCACTGAAGA GCGCTGGGAG TTACTCGAGG ATGACACTTC
GGCCGATGAG GTACAAGTTT CTTCTATTTG TTTTGGAATA
AAGTGTAATC GCCGTGCTTA ATGATTTTCC CACAATCGAT
CAGCAGGATA AGGAGATTGA TCTGCCAGAG TCCATTCCTT
TATCTGACAT GTGAGTAAAT CGGTTCATTA CTGTGATCTG
TGTAAAGTTG CAATCTTGAT CTTCTATGGT ATTAAAGGTA
ATAGTCTATT CCGGTTCTTA TGATGTTGCA GAATGCAGAG
GCAGAAAGTT AAGAAAAGCA AAAACGTGGC AGTGTCTGTG
GAACCGACTA GTTCCTCAGG TGTAAGGTGT GTGAGAATTT
ACTAAAATTC AAGTTATTGT TTATATGAAA TTTTGATGAT
GACTTGTTCT GAGAGGATTG GCGTGTATAT TGATGGTGAT
AGATCCTCAA GTAGAACACT TATGAAGAAG GATTGTGGCA
AAAGGTTGAA TAAACAAGTT GAAAAAACAA GAGAAGGAAA
GAATCTAAGA TCGTTAAAAG AGTTGAATGC TGAAACTGAC
AGGACAGCAG AAGAGCAGGA AGTGAGTCTA GAAGCTGAAT
CTGATGACAG AAGCGAAGAG CAGGAATACG AAGATGATTG
TAGCGATAAG AAAGAACAAT CTCAGGACAA AGGTGTAGAG
GCTGAAACTA AGGAAGAAGA GAAACAATAT CCAAATTCAG
AGGGTGAGAG TGAAGGAGAG GACTCAGAGT CAGAGGAAGA
GCCGAAATGG AGAGAAACAG ATGATATGGA GGATGATGAA
GAAGAAGAAG AAGAAGAGAT TGATCATATG GAGGATGAAG
CAGAAGAAGA GAAAGAAGAG GTTGATGATA AAGAGGCAAG
CGCAAACATG TCTGAGATTG AGAAAGAAGA AGAAGAAGAA
GAAGAAGATG AAGAGAAGAG AAAGTCATGA AGGAGTTACA
TAGAGTTAGA GCATTGTAAG CTAAAACCAT TTCAGAAAGA
TTCTTTCTGC TTAGACGCTC TGGTTTATCT TTCTTAGTAG
ATTTGTTGAT ATTGAACCAA GTTTTAGATG AGGTCACCTG
GTTTGTGTTT GTGTCTTGA

Two independent T-DNA insertional mutants, lrt1-1 and lrt1-2 were identified in the SALK collection. These mutants were obtained from the Arabidopsis stock center and confirmed by genotyping. The two independent mutant alleles were characterized as null for the presence of the full-length gene transcript. The insertion sites were identified by DNA sequencing.

This locus was also named PO76/PDS5D and is annotated in public databases as a cohesin homologue; however, in studies with individual mutants, there was no evidence to suggest that this specific homologue had a functional involvement in cell division (Pradillo et al., 2015). However, upon closer inspection at the cellular level of these T-DNA insertional mutants, the leaves of both mutants had a preponderance of lipid droplets in their cells. Hence, this gene locus was more aptly designated Lipid Droplet Regulatory- Tudor Domain Containing (LRT1) gene.

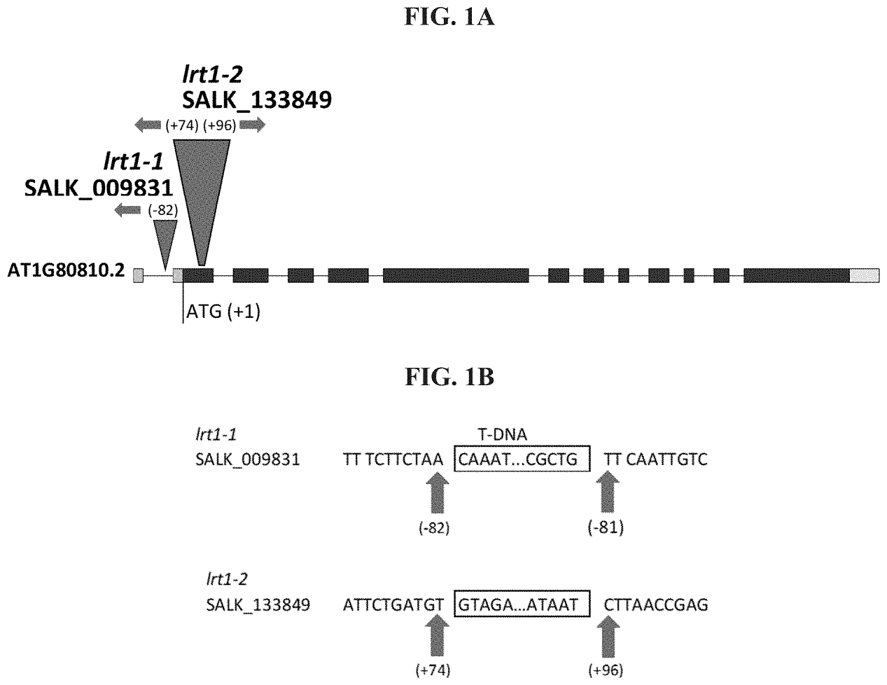

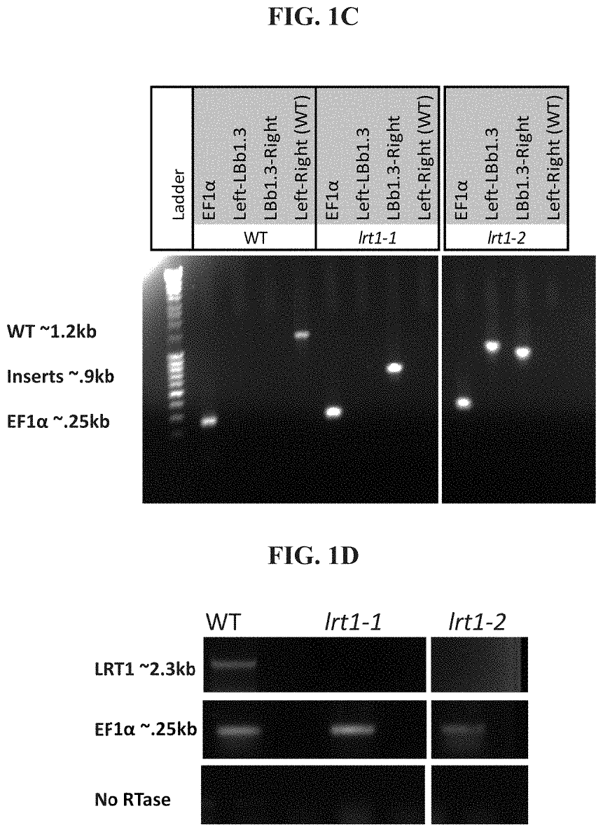

FIG. 1A shows an intron, exon map of the LRT1 gene showing the locations of the T-DNA inserts, where the light shaded portions represent UTR, the dark shaded portions represent coding regions, and the lines represent introns. T-DNA inserts are indicated by arrows at the insertion sites. The locations of the inserts (shown in parentheses) are calculated from the Adenine in the start ATG codon. lrt1-1 contains one insertion following the −82 nucleotide, lrt1-2 contains back to back insertions, replacing nucleotides between +74 and +96.

FIG. 1B shows the sequence of the insertion sites. The white boxes indicate the T-DNA insertion. Directions of the insertions are indicated in FIG. 1A.

The primers used to test for the T-DNA insertions are shown below:

SALK_009831 Left Primer
(SEQ ID NO: 2)
TTCCATTGACGAAAAGACCAG
SALK_009831 Right Primer
(SEQ ID NO: 3)
GAATCACCCGAAAGCTCTCTC
SALK_133849 Left Primer
(SEQ ID NO: 4)
AGAACCTTCTCCGCAAGACTC
SALK_133849 Right Primer
(SEQ ID NO: 5)
TGTTGGATTTGACCAGCTTTC
SALK_T-DNA insert LBb1.3
(SEQ ID NO: 6)
ATTTTGCCGATTTCGGAAC

FIG. 1C shows a PCR analysis showing the presence of the T-DNA inserts in the genomic DNA of the mutant lines. EF1α was used as positive control, and the left and right primer sequences as recommended by the Salk Institute for the respective lines as shown above were used. The insert primer was LBb1.3. Wild type tissue showed amplification of the control and undisrupted lanes only. The mutant line SALK_009831 (lrt1-1) showed the presence of a single insert, but no undisrupted gene. The mutant line SALK_133849 (lrt1-2) showed double insertions, but no undisrupted gene.

FIG. 1D shows RT-PCR showing expression of full length LRT1 in WT tissues, but none in SALK mutant lines, confirming that the T-DNA inserts disrupt normal gene expression. EF1α was run as a positive control. No Rtase was run with EF1α primers to verify absence of DNA.

### EXAMPLE 1

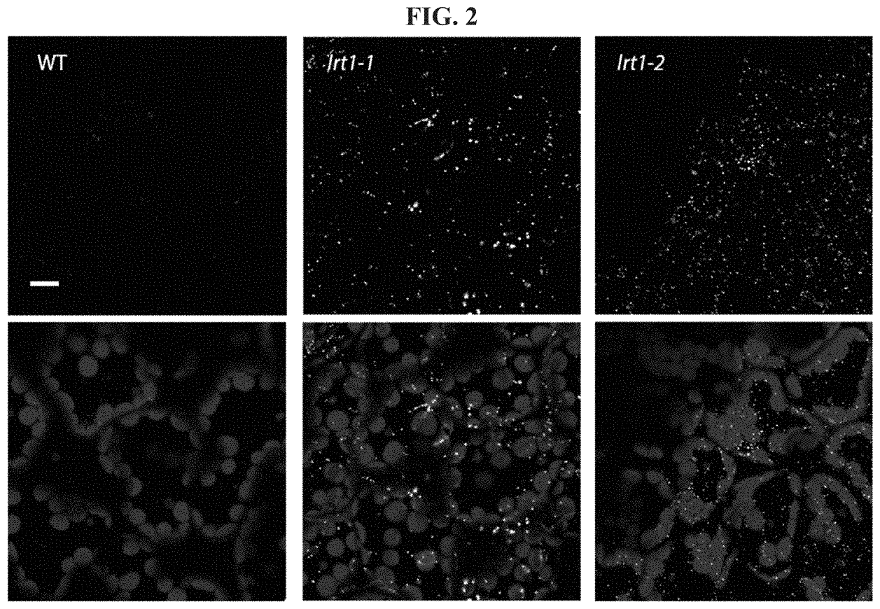

Leaves of mature Arabidopsis thaliana plants in both mutants accumulate abnormally large numbers of lipid droplets (LDs) in the cytoplasm compared with wild type (Columbia-0). LDs can be stained with BODIPY493/503, a neutral lipid specific stain. FIG. 2 shows confocal micrographs of representative images of leaf areas of 28-d-old plants. BODIPY stained LDs are shown in gray scale (top) with very few LDs visible in wild-type leaves. In the bottom images, the BODIPY-stained LDs were false-colored and merged with chlorophyll auto-fluorescence to show that they are located outside of chloroplasts in the cytoplasm. LRT1: Lipid Droplet Regulatory Tudor Domain Containing protein 1.

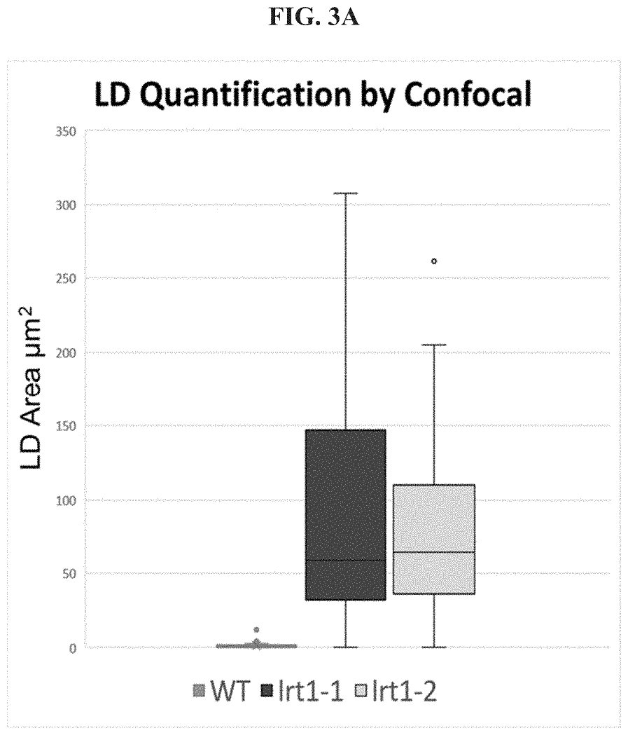

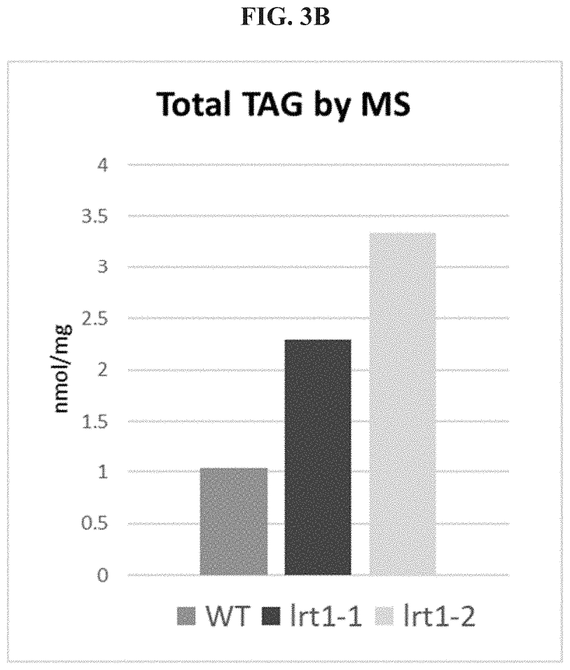

FIG. 3A shows quantification of the significant increase in lipid droplet area in mutant leaves (monitored by fluorescence microscopy and analyzed by Image J freeware). For the confocal analysis, data was drawn from the total area of LDs found on a z stack projection of 100×100×10 μm from leaf tissue of 4 week old plants (n=30). Tissue levels of storage lipids (triacylglycerols, TAGs) were significantly elevated in leaves of both LRT1 mutants, lrt1-1 and lrt1-2, as quantified by mass spectrometry. FIG. 3B shows a mass spec analysis where each bar represents quantities obtained from 3 biological replicates. Lipids were extracted from leaf tissue of 4 week old plants. This increase in measurable neutral lipid was consistent with the visible increases in BODIPY-stained neutral lipid structures in the confocal microscopy images of the leaves of these two mutants shown in FIG. 2.

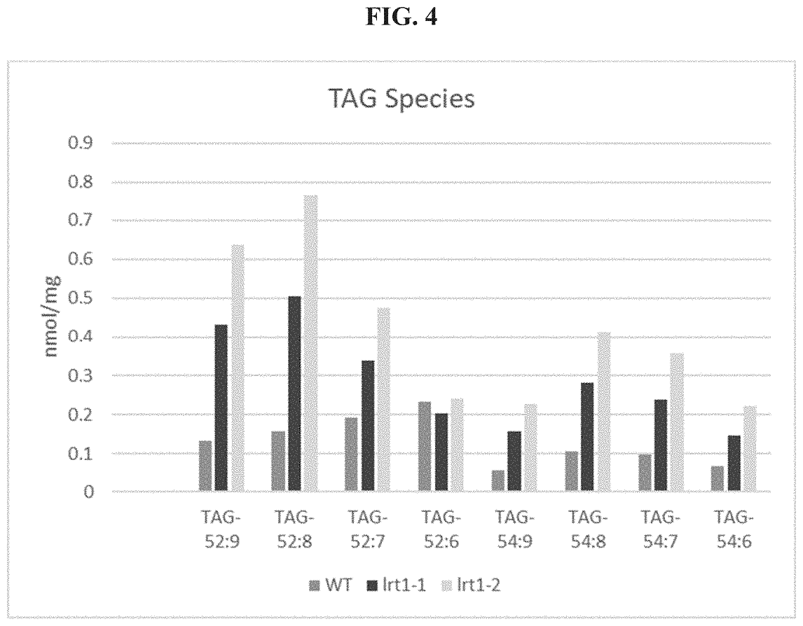

The TAG species in mutant leaves are also more highly unsaturated as compared to the wild type. FIG. 4 shows profiles of storage lipid (TAG) individual molecular species, demonstrating that omega-3 polyunsaturated fatty acids were mostly enriched in the storage lipids of the mutants. The numerical designation indicates total number of carbon atoms in the acyl chains of the TAGs and the total number of double bonds. For example TAG 52:9 is a triacylglycerol molecular species with the acyl composition of 16:3/16:3/18:3.

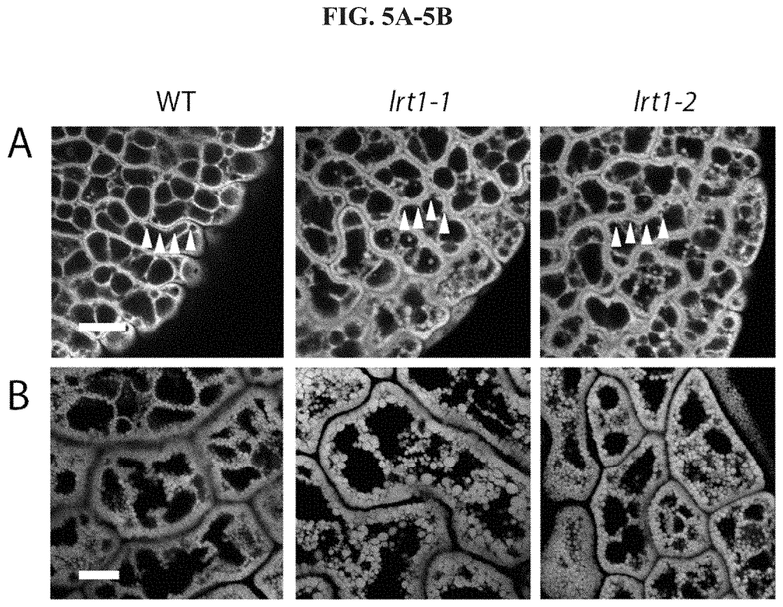

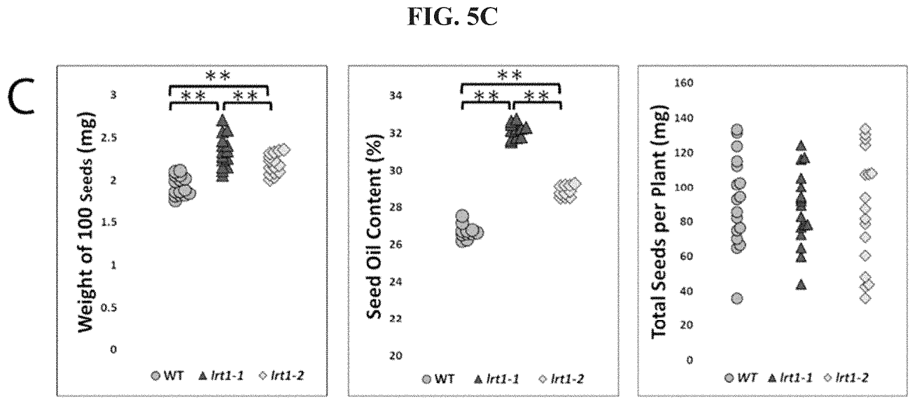

In addition to elevated neutral lipids in leaves, the mutants also produced significantly larger seeds with significantly higher seed oil contents. FIG. 5 shows an analysis of seed lipids in lrt mutants versus wild-type. Seeds of Arabidopsis lrt1 mutants show increased LD presence over WT. FIG. 5A shows single layer confocal laser scanning microscopy (CLSM) images of BODIPY 493/503 stained embryos. Seed embryos of lrt1-1 and lrt1-2 show a thicker layer of LDs clustered along the cell walls than do WT embryos. FIG. 5B shows Airyscan CLSM images of BODIPY 493/503 stained embryos. Images are z-stack projections of 30×30×3 μm. Scale bar 5μm. Individual LDs are larger in the lrt1-1 and lrt1-2 mutants than those found in WT. FIG. 5C shows (Left) Graph showing the weight of 100 seeds, n=15 for all genotypes and (Middle) seed oil content measured by time-domain, 1H-NMR, WT and lrt1-1 n=11, lrt1-2 n=10. Each point represents an independent replicate. Significance determined by One-way ANOVA (** P<0.01) with Bonferroni and Holm post-hoc testing. FIG. 5C (Right) shows total seed weight, n=17 plants for each genotype. As seen in FIG. 5C, seeds of lrt1 mutants have significantly increased weight and oil content over WT. lrt1 mutants show increased seed oil content over WT seeds. Total seeds per plant as determined by weight did not vary significantly.

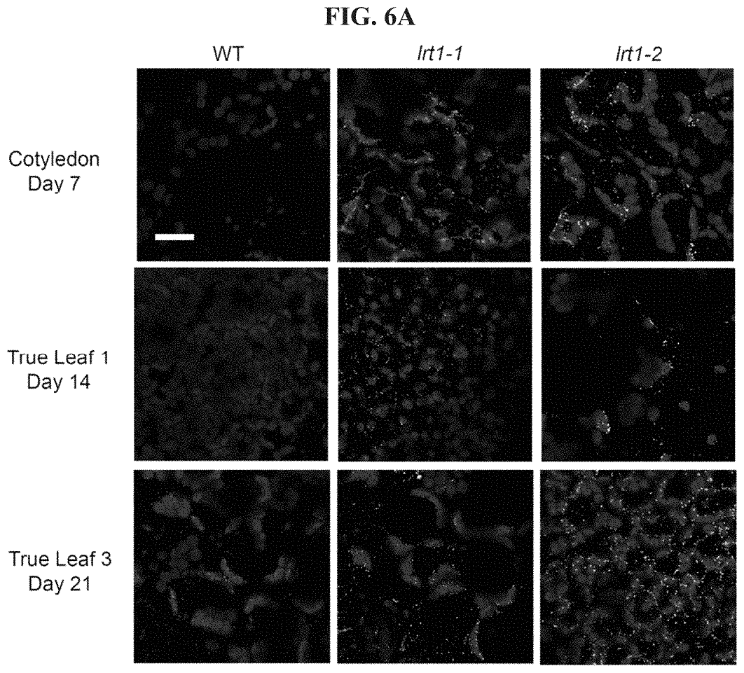

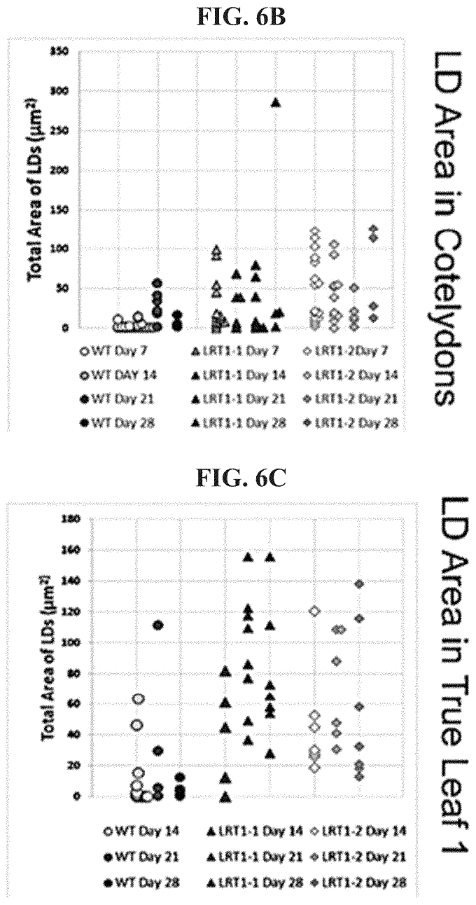

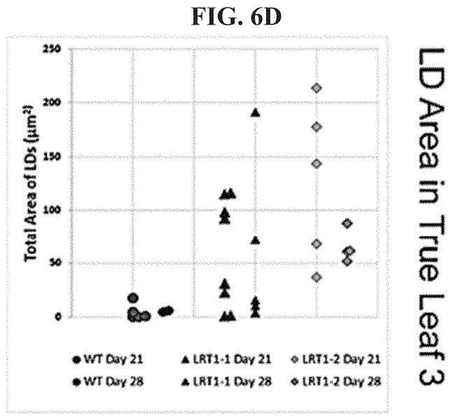

Plants were examined at several stages of development, and the increased lipid droplet phenotype was visible and quantifiable in early stages of seedling development (green cotyledons) and well as in leaves later in development. The elevated lipid droplet phenotype in leaves is evident in green cotyledons of seedlings and in sequential leaves developing plants in the Arabidopsis lrt1 mutants. FIG. 6A shows confocal images of portions of leaves as was shown in FIG. 2. BODIPY-stained LDs were false-colored and chloroplasts were marked by autofluorescence. FIG. 6B shows graphs quantifying LDs in multiple images from cotyledons and true leaves (FIG. 6C and 6D) during 28 days of development. The data demonstrate that the LD phenotype is present throughout the life of the plant.

It is clear that the absence of expression of the LRT gene leads to plants with an overall increase in lipid storage in both vegetative tissues and in seeds. This is consistent with a role for this gene in suppression of oil accumulation in plants.

### EXAMPLE 2

It seemed possible that the synthesis of additional energy-rich lipids in plant tissues might compromise plant growth in some way. However, growth parameters, flowering time, and photosynthetic rates were examined and no deleterious effects were found.

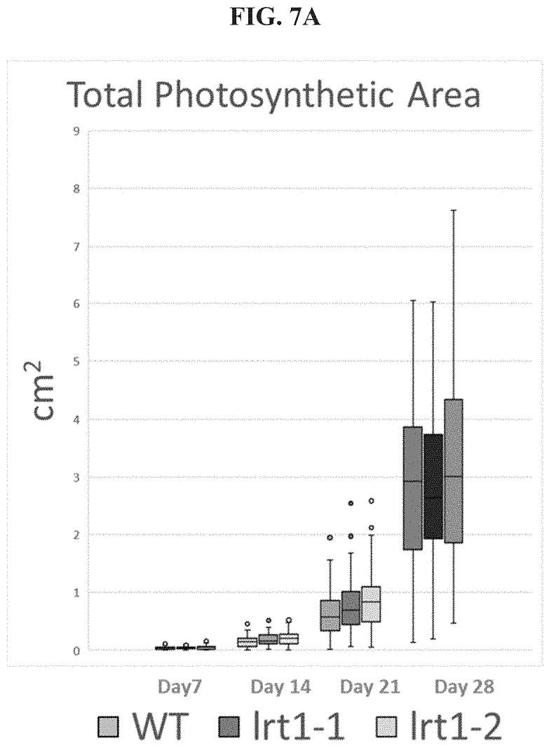

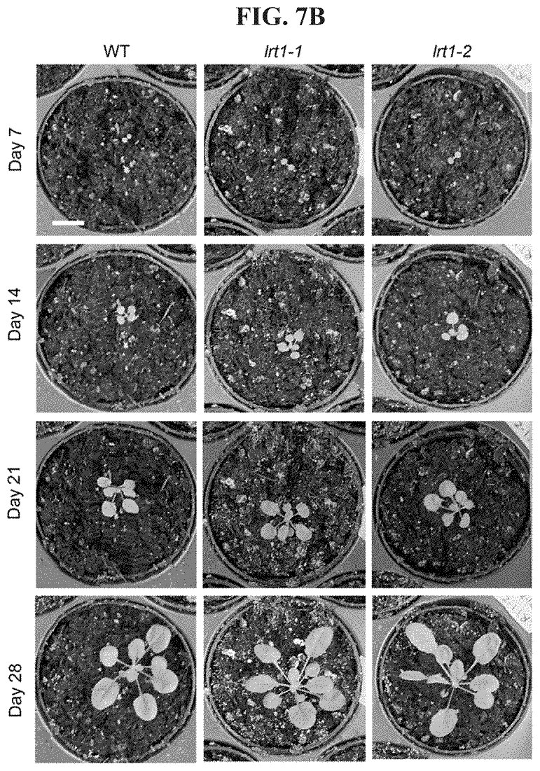

FIG. 7A shows total photosynthetic leaf areas quantified over 28 days of development for wild type and the two mutant plants. The presence of LDs in leaves did not affect growth rate or plant size. FIG. 7B shows photographs of plants over 28 days. There were no significant differences among genotypes, indicating that enhanced lipid production in leaves does not interfere with normal growth.

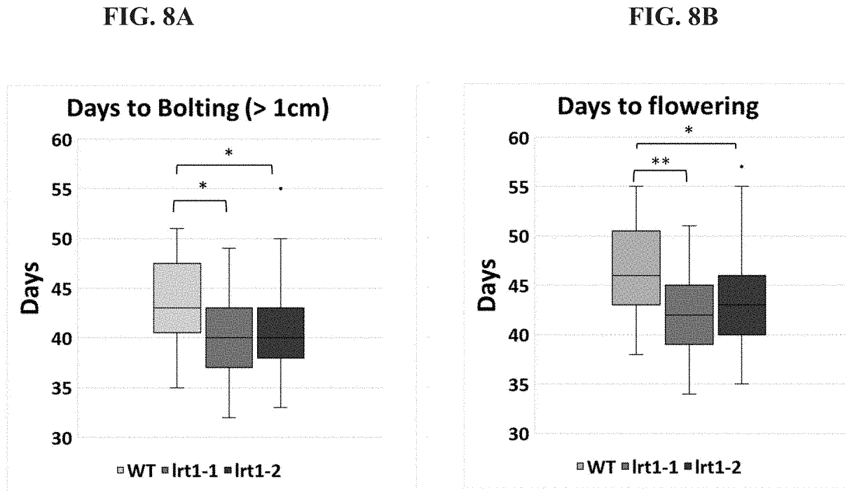

FIG. 8A shows days to bolting (<1 cm) and FIG. 8B shows days to first flower opening for wild type and mutants. Both mutants in LRT1 appeared to flower earlier than wild-type by a couple of days. This was reflected by days to “bolting” and days to first open flower. In the data, n=17 and for both figures, significance was determined by one-way ANOVA with Bonferroni and Holm post-hoc testing. (* P<0.05, ** P<0.01).

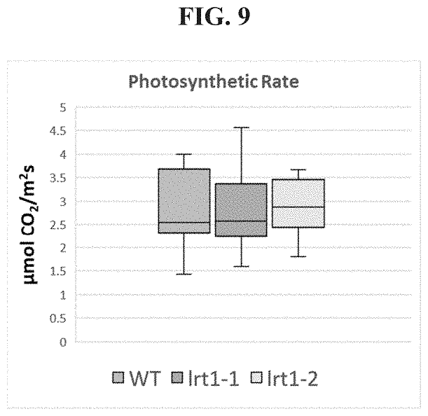

FIG. 9 shows photosynthetic rate calculated for the wild type and both mutants. Rates of CO2 incorporation per unit leaf area were measured by a LiCOR infrared gas analyzer instrument, the LI-COR LI-6400XT Portable Photosynthesis System. Each plant was measured in triplicate and the average of triplicate readings was normalized against total leaf area as measured with ImageJ software. Photosynthetic rates were roughly equivalent among genotypes, suggesting that increased storage lipids in leaves did not affect the capacity for photosynthesis.

Mutant plants looked mostly indistinguishable from wild-type plants at the morphological and physiological levels. Only the cellular increase in storage lipids distinguished these mutant plants from wild-type. This bodes well for strategies designed to inactivate this gene for energy densification of crop plants.

What is claimed is:

1. A method for producing a modified plant having increased oil content or increased cytosolic lipid droplet (LD) volume in cells of the plant compared to an unmodified plant of the same species, comprising:
introducing a mutation into a gene in plant cells to produce modified plant cells comprising a mutated gene, wherein the gene is LRT1, and wherein the mutation is a T-DNA insertion;
cultivating the modified plant cells to produce a modified plant, wherein substantially all cells of the modified plant comprise the mutated gene, and wherein cells of the modified plant accumulate oils or cytosolic lipid droplets (LDs) in increased amounts compared to cells of unmodified plants of the same species.

2. The method of claim 1, wherein the gene has a sequence comprising SEQ ID NO:1.

3. The method of claim 1, wherein the plant is an Arabidopsis plant.

4. The method of claim 1, wherein the plant is a canola, Camelina, soybean, sunflower, safflower, cotton, palm, coconut, or peanut plant.

5. A modified plant having increased oil content or increased cytosolic lipid droplet (LD) volume in cells of the plant compared to an unmodified plant of the same species, wherein substantially all cells of the plant comprise a gene having a mutation, wherein the gene is LRT1, wherein the mutation is a T-DINA insertion, and wherein cells of the modified plant accumulate oils or cytosolic lipid droplets (LDs) in increased amounts compared to cells of unmodified plants of the same species.

6. The modified plant of claim 5, wherein the gene has a sequence comprising SEQ ID NO:1.

7. A seed of the modified plant of claim 5.

8. The modified plant of claim 5, wherein the modified plant is an Arabidopsis plant.
